SEQUENCE LISTING (40 sequences)

SEQ ID NO: 1 - DNA, 12 bp, Artificial Sequence
  Synthetic Oligonucleotide V2.
cttgcctcttgg

SEQ ID NO: 2 - DNA, 16 bp, Artificial Sequence
  Synthetic Oligonucleotide V3.
catttggctcccagcc

SEQ ID NO: 3 - DNA, 18 bp, Artificial Sequence
  Synthetic Oligonucleotide V4.
ggttgtctgaagtagcac

SEQ ID NO: 4 - DNA, 17 bp, Artificial Sequence
  Synthetic Oligonucleotide V5.
gtggggtctcttcttcc

SEQ ID NO: 5 - DNA, 12 bp, Artificial Sequence
  Synthetic Oligonucleotide V6.
cctcatgccatc

SEQ ID NO: 6 - DNA, 17 bp, Artificial Sequence
  Synthetic Oligonucleotide V7.
gttggtgttgtccttcc

SEQ ID NO: 7 - DNA, 18 bp, Artificial Sequence
  Synthetic Oligonucleotide V8.
ttgcagtaggctgaagcg

SEQ ID NO: 8 - DNA, 16 bp, Artificial Sequence
  Synthetic Oligonucleotide V9.
tatcttcttccaagcc

SEQ ID NO: 9 - DNA, 18 bp, Artificial Sequence
  Synthetic Oligonucleotide V10.
ctgggatgaaggtcctgc

SEQ ID NO: 10 - DNA, 20 bp, Artificial Sequence
  Synthetic Oligonucleotide 5-V2.
actagtgctcatcaaagtgg

SEQ ID NO: 11 - DNA, 20 bp, Artificial Sequence
  Synthetic Oligonucleotide 5-V3.
ggtatttgaagacgtactgg

SEQ ID NO: 12 - DNA, 20 bp, Artificial Sequence
  Synthetic Oligonucleotide 5-V4.
ccgtggtgtggttgaaatgg

SEQ ID NO: 13 - DNA, 20 bp, Artificial Sequence
  Synthetic Oligonucleotide 5-V5.
gccatttctgtctacattgg

SEQ ID NO: 14 - DNA, 20 bp, Artificial Sequence
  Synthetic Oligonucleotide 5-V6
actaggagttgcctggatgg

SEQ ID NO: 15 - DNA, 20 bp, Artificial Sequence
  Synthetic Oligonucleotide 5-V7.
ggtatgagctgaggctgtgg

SEQ ID NO: 16 - DNA, 20 bp, Artificial Sequence
  Synthetic Oligonucleotide 5-V8.
atgactggagtccatattgg

SEQ ID NO: 17 - DNA, 20 bp, Artificial Sequence
  Synthetic Oligonucleotide 5-V9.
ctgagaattactctgcttgg

SEQ ID NO: 18 - DNA, 20 bp, Artificial Sequence
  Synthetic Oligonucleotide 5-V10.
tgtgacatcattcctattgg

SEQ ID NO: 19 - DNA, 20 bp, Artificial Sequence
  Synthetic Oligonucleotide 5-16.
gaatgtgtcttggtctctgg

SEQ ID NO: 20 - DNA, 20 bp, Artificial Sequence
  Synthetic Oligonucleotide V2-16.
tgtgtcttggtctccagcca

SEQ ID NO: 21 - DNA, 20 bp, Artificial Sequence
  Synthetic Oligonucleotide V3-16.
tgtgtcaaggtctctggtgc

SEQ ID NO: 22 - DNA, 20 bp, Artificial Sequence
  Synthetic Oligonucleotide V4-16.
gaatgtgtcttggtctccag

SEQ ID NO: 23 - DNA, 20 bp, Artificial Sequence
  Synthetic Oligonucleotide V5-16.
gaatgtgtcttggtctcttg

SEQ ID NO: 24 - DNA, 20 bp, Artificial Sequence
  Synthetic Oligonucleotide V6-16.
aatgtgtcttggtctccagc

SEQ ID NO: 25 - DNA, 20 bp, Artificial Sequence
  Synthetic Oligonucleotide V7-16.
gaatgtgtcttggtctccca

SEQ ID NO: 26 - DNA, 20 bp, Artificial Sequence
  Synthetic Oligonucleotide V8-16.
aatgtgtcttggtctcgcgt

SEQ ID NO: 27 - DNA, 20 bp, Artificial Sequence
  Synthetic Oligonucleotide V9-16.
gaatgtgtcttggtctctgc

SEQ ID NO: 28 - DNA, 20 bp, Artificial Sequence
  Synthetic Oligonucleotide V10-16.
aatgtgtcttggtctcctga

SEQ ID NO: 29 - DNA, 20 bp, Artificial Sequence
  Synthetic Oligonucleotide V2-V3.
atttgaagacgtaccagcca

SEQ ID NO: 30 - DNA, 20 bp, Artificial Sequence
  Synthetic Oligonucleotide V3-V4.
tggtgtggttgaaatggtgc

SEQ ID NO: 31 - DNA, 20 bp, Artificial Sequence
  Synthetic Oligonucleotide V4-V5.
gccatttctgtctacatcag

SEQ ID NO: 32 - DNA, 20 bp, Artificial Sequence
  Synthetic Oligonucleotide V5-V6.
actaggagttgcctggattg

SEQ ID NO: 33 - DNA, 20 bp, Artificial Sequence
  Synthetic Oligonucleotide V6-V7.
gtatgagctgaggctgcagc

SEQ ID NO: 34 - DNA, 20 bp, Artificial Sequence
  Synthetic Oligonucleotide V7-V8.
atgactggagtccatatcca

SEQ ID NO: 35 - DNA, 20 bp, Artificial Sequence
  Synthetic Oligonucleotide V8-V9.
ctgagaattactctgctgcg

SEQ ID NO: 36 - DNA, 20 bp, Artificial Sequence
  Synthetic Oligonucleotide V9-V10.
tgtgacatcattcctattgc

SEQ ID NO: 37 - DNA, 40 bp, Artificial Sequence
  Synthetic Oligonucleotide T7-E5.
ttgtaatacgactcactatagggacagtccctggatcacc

SEQ ID NO: 38 - DNA, 44 bp, Artificial Sequence
  Synthetic Oligonucleotide T7-EV6.
ttgtaatacgactcactatagggcaactcctagtagtacaacgg

SEQ ID NO: 39 - DNA, 24 bp, Artificial Sequence
  Synthetic Oligonucleotide R-E16.
gtttgctccaccttcttgactccc

SEQ ID NO: 40 - DNA, 19 bp, Artificial Sequence
  Synthetic Oligonucleotide R-V6.
gtactactaggagttgcct

(19) United States
(12) Patent Application Publication
(10) Pub. No.: US 2003/0170637 A1
(43) Pub. Date: Sep. 11, 2003

(54) Method of analyzing mRNA splice variants

(76) Inventors: Michael C. Pirrung, Cardiff, CA (US); Hyunsoo Kim, Chapel Hill, NC (US)

Correspondence Address: MYERS BIGEL SIBLEY & SAJOVEC, PO BOX 37428, RALEIGH, NC, 27627, US

(21) Appl. No.: 10/092,208

(22) Filed: Mar. 6, 2002

(51) Int. Cl. C12Q 1/68; C12P 19/34

(52) U.S. Cl. 435/6; 435/91.2

(57) ABSTRACT

A method for determining the exons present in a potentially variantly spliced mRNA is carried out by: (a) providing a potentially variantly spliced mRNA; (b) providing an array, the array comprising a plurality of different primers immobilized on a solid support at distinct locations thereon, with each of the plurality of different primers selectively hybridizing to a corresponding one of the plurality of exons potentially within the mRNA; (c) contacting the mRNA to the array that a duplex is formed between each different primer and each corresponding exon if the corresponding exon is included in the mRNA; (d) subjecting the duplexes to a primer extension reaction so that the primers in the duplexes are extended with at least one labeled base; and then (e) detecting the presence or absence of the at least one labeled base in each of the plurality of primers, the presence of the at least one labeled base indicating the presence of the exon to which the primer selectively binds in the potentially variantly spliced mRNA. Apparatus and arrays for carrying out the invention are also disclosed

### STATEMENT OF FEDERAL SUPPORT

This invention was made possible with government support under grant number NIH GM 46720 from the National Institutes of Health and NSF EIA-0086015 from the National Science Foundation. The United States government has certain rights to this invention.

### FIELD OF THE INVENTION

This invention concerns methods, systems, and materials useful for the analysis of variant exon splicing in mature mRNAs.

### BACKGROUND OF THE INVENTION

The completion of the human genome sequence (Venter, et al. (2001) Science 291:1304) has revealed that the total number of genes is less than had been earlier estimated, perhaps 30,000. Because this value is also significantly less than the number of human cDNAs (Claverle (2001) Science 291:1255) that have been identified, perhaps 120,000, it is apparent that a portion of protein sequence diversity is due not to genomic sequences but to alternative splicing of initial RNA transcripts. Of 14,000 known human genes, >40% possess multiple variant spliced forms (Sakharkar, et al. (2000) Bioinformatics 16:1151-2; Sakharkar, et al. (2000) Nucleic Acids Res. 28:191-2). Understanding of the biological functions of the genome will therefore benefit from methods to examine each exon in variantly spliced mRNAs. Ideally, such methods would permit many RNAs to be examined simultaneously. Microarrays are ideal for highly parallel analysis, but microarray techniques with fidelity adequate to analyze complex mRNA for individual exons are unknown.

Many valuable microarray methods have been devised for studying levels of mRNAs or cDNAs in parallel (Hughes and Shoemaker (2001) Curr. Opin. Chem. Biol. 5:21-25). Hybridization data, which has an analog form, can be used for differential gene expression analysis (Schena, et al. (1995) Science 270:467-470) e.g., the comparison of RNA transcript levels under varying conditions/treatments. Hybridization to “exon arrays” has also been used in the annotation of the human genome (Shoemaker, et al. (2001) Nature 409:922-7). As described above, the efficient analysis of splicing variants relies upon digital information. The APEX (arrayed primer extension) method (Shumaker, et al. (2001) Bioorg. Med. Chem. 9:2269) provides high-fidelity, essentially digital detection of nucleic acid sequences with high parallelism. APEX relies on single-nucleotide extension of microarrays of DNA primers complementary to a template by a polymerase. Further, APEX has been used for the solution of Boolean problems, including those of the NP-complete class (Pirrung, et al. (2000) J. Am. Chem. Soc. 122:1873). However, none of these techniques has provided a way to analyze variant exon splicing in mature mRNAs.

### SUMMARY OF THE INVENTION

The present invention describes the use of APEX microarray RNA analysis (Pirrung, et al. (2000) J. Am. Chem. Soc. 122:1873; Tollett, et al. (1997) Am. J. Hum. Gen. 61(S):1322) to analyze variantly spliced mRNAs. The application of APEX to RNA templates and surface-bound DNA primers requires single-nucleotide extension by a reverse transcriptase (RT). Earlier investigations of RNA primer extension were conducted but have never been applied to a microarray nor had an optimal RT enzyme been discovered (Pirrung, et al. (2001) Bioorg. Med. Chem. Lett. 11:2437).

A first aspect of the present invention is a method for determining the exons present in a potentially variantly spliced mRNA. The method comprising the steps of: (a) providing a potentially variantly spliced mRNA, the mRNA encoded by a DNA, the DNA comprising a plurality of exons, each of which plurality of exons may or may not be included in the mRNA; (b) providing an array, the array comprising a plurality of different primers immobilized on a solid support at distinct locations thereon, with each of the plurality of different primers selectively hybridizing to a corresponding one of the plurality of exons (e.g., under predetermined hybridization conditions) to form a duplex therebetween; (c) contacting the mRNA to the array (e.g., under the predetermined hybridization conditions) so that a duplex is formed between each different primer and each corresponding exon if the corresponding exon is included in the mRNA; (d) subjecting the duplexes to a primer extension reaction so that the primers in the duplexes are extended with at least one labeled base; and then (e) detecting the presence or absence of the at least one labeled base in each of the plurality of primers, the presence of the at least one labeled base indicating the presence of the exon to which the primer selectively binds in the potentially variantly spliced mRNA.

A second aspect of the present invention is an array useful for determining the exons present in a potentially variantly spliced mRNA as described above. Such an array comprises (a) a solid support; and (b) a plurality of different primers as described above immobilized on the solid support at distinct locations thereon. In one preferred embodiment, the primers are immobilized to the solid support by the 5′ end thereof, so that the 3′ end is free for primer extension. In an illustative embodiment of the invention, the mRNA is CD44 mRNA (and thus the primers hybridize to different CD44 exons).

A third aspect of the present invention is a system for determining the exons present in a potentially variantly spliced mRNA with a plurality of different primers, as described above. The system comprises (a) a detector (e.g., a fluorescence detector) for detecting the presence or absence the labeled base from each of the plurality of primers; (b) a signal generator (e.g, an analog-to digital converter or other suitable interface) operatively associated with the detector for generating a plurality of values, each of the values indicating the presence or absence of each of the exons in the mRNA from the detected presence or absence of the labeled base from each of the plurality of primers; and (c) a processor (e.g., an appropriately programmed computer or cpu or other suitable device) operatively associated with the signal generator for generating a code representing the exons present in the mRNA from the plurality of generated values. In general, in a preferred embodiment, such a system will also include a storage device and display operatively associated with the processor.

A fourth aspect of the present invention is a method for distinguishing splice variants in a mixed mRNA sample. The method comprises the steps of: (a) providing a mixed mRNA sample, the mixed mRNA sample comprising a plurality of splice variants, each one of the plurality of splice variants containing a distinct exon-exon junction not found in each other of the plurality of splice variants; (b) providing an array, the array comprising a plurality of different primers immobilized on a solid support at distinct locations thereon, with each of the plurality of different primers selectively hybridizing to a corresponding one of the distinct exon-exon junctions (e.g., under predetermined hybridization conditions) to form a duplex therebetween; (c) contacting the mixed mRNA sample to the array (e.g., under the predetermined hybridization conditions) so that a duplex is formed between each different primer and each corresponding splice variant if the corresponding splice variant is included in the mixed mRNA sample; (d) subjecting the duplexes to a primer extension reaction so that the primers in the duplexes are extended with at least one labeled base; and then (e)detecting the presence or absence of the labeled base in each of the plurality of primers, the presence of the at least one labeled base indicating the presence of the splice variant to which the primer selectively binds in the mixed mRNA sample.

A fifth aspect of the present invention is an array useful for distinguishing splice variants in a mixed mRNA sample as described above. The array comprises: (a) a solid support; and (b) a plurality of different primers as described above immobilized on the solid support at distinct locations thereon. In a preferred embodiment, wherein each of a plurality of the primers is directed to an exon-exon junction of a common exon fused or spliced to one of a variety of possible variable exons, each of the plurality of primers contains a common primer segment coupled to a variable primer segment, with the common primer segment corresponding to a common exon segment and being the same among the plurality of primers, and with the variable primer segment corresponding to a variable exon segment and being different among the plurality of primers. In one embodiment, the common primer segments are from 8 to 50 nucleotides in length or more, and are positioned at the 5′ end of the primers (for attachment to the solid support). In one embodiment, the variable primer segments are from 2 or 3 to 6 or 7 nucleotides in length, and are positioned at the 3′ end of the primers (where they are free for extension with the at least one labeled base).

A fifth aspect of the present invention is a system for distinguishing splice variants in a mixed mRNA sample with a plurality of different primers, as described above. In general, such a system comprises (a) a detector such as described above for detecting the presence or absence of the labeled base from each of the plurality of primers; (b) a signal generator such as described above operatively associated with the detector for generating a plurality of values, each of the values indicating the presence or absence of each of the splice variants in the mixed mRNA sample from the detected presence or absence of the labeled base from each of the plurality of primers; and (c) a processor such as described above operatively associated with the signal generator for generating a determination of the splice variants present in the mixed mRNA sample from the plurality of generated values. Again, such a system will generally include a storage device and a display device operatively associated with the processor.

The foregoing and other objects and aspects of the present invention are explained in detail in the specification set forth below.

### BRIEF DESCRIPTION OF THE DRAWINGS

FIG. 1 graphically depicts that the CD44 locus comprises 20 exons, of which exons 6-15 are omitted in normal splicing. The variably spliced exons have alternative descriptors v2-v10 (in humans, v1 has an in-frame stop codon and must be silent). If variable exons are included in the mRNA, up to 381 amino acids are inserted into the 270 amino acid CD44 extracellular domain.

FIG. 2 shows RNA APEX on a CD44 exon-specific microarray. The spot size is ˜80 μm. Panel A demonstrates APEX with RNA template prepared from CD44 v2-v10 produced by PCR with primers T7-exon 5 and reverse exon 16. The average signal-to-background (S/B) ratio (the spot compared to adjacent sites without primer) across all primers is 18; Panel B demonstrates APEX with RNA template prepared from CD44 v6-v10 produced by PCR with primers T7-exon v6 and reverse exon 16; Panel C shows APEX with RNA template prepared from CD44 v2-v5 produced by PCR with primers T7-exon 5 and reverse exon v6; Panel D depicts the key for Panels A-C.

FIG. 3 shows a 1% agarose gel of PCR products from a human breast tumor cDNA template. Lane 1:1-Kb ladder; lane 2: PCR products from human breast tumor cDNA template; Lane 3: PCR product from CD44 v2-v10 clone cDNA template.

FIG. 4 shows RNA APEX with a human breast tumor CD44 template. Panel A shows exon 5-variable exon border primers; Panel B shows the key for Panel A; Panel C shows variable exon-exon 16 border primers; Panel D shows the key for Panel C; Panel E shows the variable exon-variable exon border primers; Panel F shows the key for Panel E.

FIG. 5 schematically illustrates an array that may be used to carry out certain aspects of the present invention.

FIG. 6 schematically illustrates an apparatus that may be used to carry out the present invention.

### DETAILED DESCRIPTION OF PREFERRED EMBODIMENTS

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Applicants specifically intend that the disclosures of all United States patents cited herein are to be incorporated herein by reference in their entirety.

1. Definitions.

“Exon” as used herein refers to a portion of a gene that is or may be expressed. The exon may exist as a segment of a transcript within genomic DNA, mRNA (including pre-mRNA and mature mRNA), or a cDNA. Exons within a particular genomic DNA or pre-mRNA may be interrupted by intervening sequences called introns which are not expressed. Introns may be removed from a pre-mRNA by a process known as splicing to form the mature mRNA.

RNA or mRNA used to carry out the present invention may be naturally occurring or polymerized from a source such as a cDNA or genomic DNA. The mRNA may be provided intact or may be provided in a fragmented form, either because the natural material in the sample exists as fragments or by carrying out a fragmenting step on the RNA prior to contacting it to a probe or solid support.

“Variantly spliced mRNA” refers to mRNA which is encoded by the same genomic DNA, but which may vary in nucleotide sequence and/or the encoded protein due to differences in which exons are included in the mature mRNA. The process or processes by which different combinations of exons from the same genomic DNA are spliced together to form different possible combinations of mature mRNA is not entirely known, but is generally referred to as alternative splicing or variant splicing.

“Splice variant” or “splice variants” refers to different mRNAs originating from the same genomic DNA, but having different combinations of exons therein due to alternative splicing.

“Exon-exon” junction as used herein refers to a segment of mRNA that consists of one or more oligonucleotides contributed by one exon coupled to one or more nucleotides contributed by a separate exon (i.e., with the intervening intron, or intervening intron(s) and exon(s), removed). The particular “size” of the exon-exon junction is not critical, but may be considered in conjunction with the size of the probe or primer which is designed to bind thereto.

“Distinct exon-exon junctions” herein refers to exon-exon junctions which differ from one another. In some, but not all, embodiments of the invention, distinct exon-exon junctions share one common exon and thus have a portion in common.

“Array” refers to an article of manufacture containing a plurality of primers used to study molecular binding interactions, such as between oligonucleotides. An array is typically formed from a solid support, to which a plurality of primers can be bound at distinct locations thereon.

“Primers” used herein are generally oligonucleotide primers, which may be natural or synthetic and may include modified backbone primers, such as DNA-RNA hybrids.

“Hybridization conditions” refers to the conditions under which a primer is contacted to a target or template nucleic acid, and are defined by parameters such as temperature, buffer concentration, pH, salt concentration and the like. Hybridization conditions may range from reduced stringency conditions (meaning binding of the primer to the target is favored and will tolerate one or more mismatches in the target sequence) to stringent conditions (meaning binding of the primer to the target is disfavored unless few or no mismatches are present). In general, the hybridization conditions are predetermined in the sense that they are either standardized or known for a given assay, with a given set or class of primers and a given category of target or template mRNA.

“Labeled bases” are nucleotide bases used to carry out primer extension reactions as described below labeled with a suitable detectable group such as a fluorescent group, a chemiluminescent group, a radioactive iosotope, a protein, a hapten such as digoxin, etc. In addition to such extrinsic labels, bases may be intrinsically or inherently labeled when they are different primers are each labeled with a mutually distinguishable base that can then be differentiated and/or detected by a technique such as surface plasmon resonance (SPR) microscopy. Hence the term “detectable base” is to be considered interchangeable with the term “labeled base” in the instant application unless intrinsic or inherent labels are expressly excluded therefrom.

A “primer extension reaction” is one which extends the primer with a labeled or detectable base as described above. Such reactions are well known and are, in general, modifications of polymerase chain reaction technology. Where as here the target or template nucleotide is mRNA, the reaction may be carried out with a reverse transcriptase.

2. mRNA Splicing and Exon Usage.

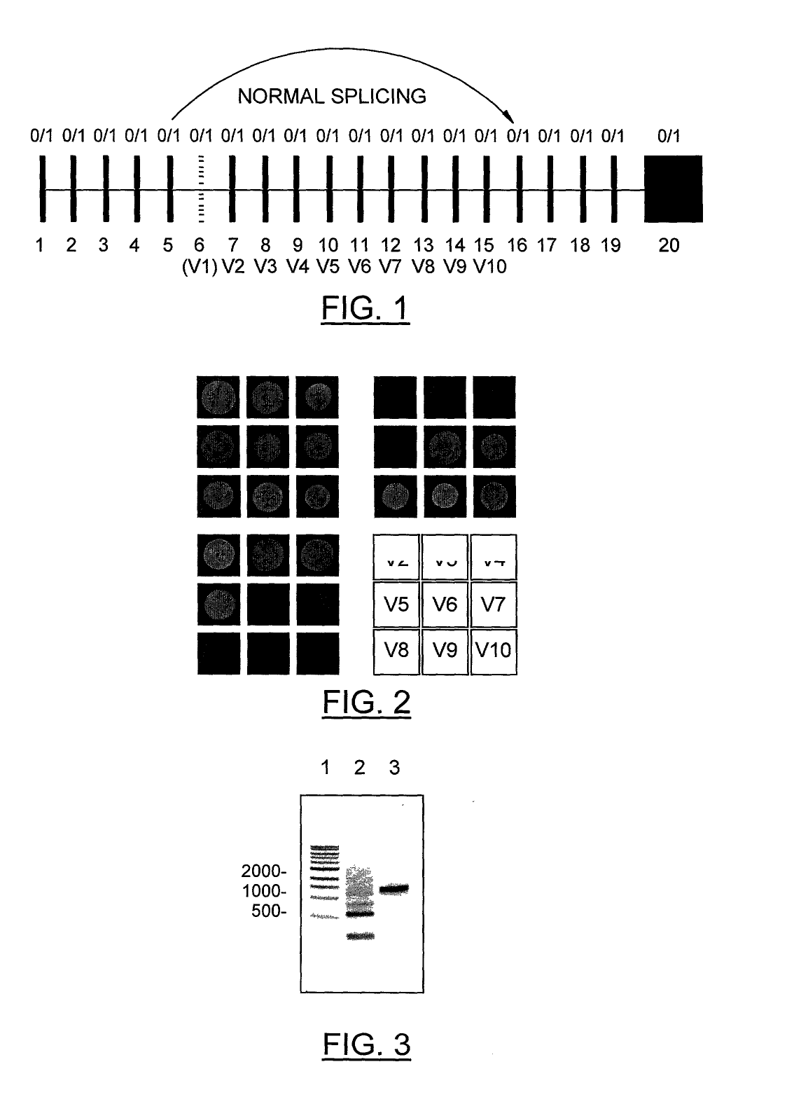

Understanding of the spliced forms of a mature mRNA begins with identification of the exon usage. The presence or absence of an exon is a Boolean variable, and the structure of an mRNA formed from a genomic sequence of n exons can be represented by an n-bit binary number. For example, CD44 is a cell adhesion molecule (Goodison et al. (1999) Mol. Pathol. 52:189-96) found on the surface of all mammalian cells. Its genetic locus (human chromosome 11p13) comprises twenty exons, ten of which are alternatively spliced (FIG. 1). Thus, a molecular code for the structure of a CD44 mRNA is simply a 20-bit number. So far as is known, exons encoding the lectin domain (1-5) and the membrane proximal, transmembrane, and cytoplasmic domains (16-20) are always included, so codes for mRNAs for all functional CD44s begin and end with 11111. The normally spliced mRNA would be coded 11111000000000011111; known variants will have one or more of the 0 s switched to 1 s. This results in >1000 (2¹⁰) possible spliced transcript sequences. Determination of the structure of a mature CD44 mRNA is thus a combinatorial analytical problem (Smith and Valcárcel (2000) Trends Biochem. Sci. 25:381-8). When multiplied by the vast number of genes, analysis of RNA splicing across the genome would become intractable (like RNA folding; Lyngso and Pedersen (2000) J. Comput. Biol 7:409-27), it may be NP-complete (Garey and Johnson. Computers and Intractability: A Guide to the Theory of NP-completeness (Freeman, San Francisco, 1979)). Gaspin, C.; Westhof, E. Adv. Mol. Bioinf. Ed: Schulze-Kremer, S. IOS Press, Amsterdam, (1994), p. 103)). The ability to generate digital information on exon splicing employing the methods and techniques described further below allows the use of this information for studying and analyzing exon splicing in greater detail.

3. Methods.

The RNA on which the present invention is carried out can be obtained from any suitable source, including both plant and animal RNA, as well as RNA of various microorganisms (e.g., yeas, bacteria). In general, the RNA is preferably of eukaryotic origin. Animal RNA may be obtained from any suitable source including but not limited to fish, birds, reptiles, insects, amphibians and mammals (including humans, monkeys, cats, dogs, rats, rabbits, etc.). In certain embodiments the invention is carried out with mammalian, particularly human, mRNA for examining splicing diseases. Such RNA may encode CD44 as in the Examples given below, or may be one of the following genes or encode one of the following proteins, as in the splicing diseases indicated below:

| Splicing diseases | gene |
|---|---|
| breast or ovarian cancer | BRCA1 and BRCA2 |
| melanoma | CDKN2A |
| melanoma | tyrosmase |
| melanoma | Amphiphysin II |
| leukemia | BAALC |
| glioblastoma | fibroblast growth factor receptor-1 |
| nervous system tumors | neurofibromatosis type I (NF1) |
| Neurodegeneration (Alzheimer's) | beta/A4 amyloid protein |
| precursor | |
| Neurodegeneration (Alzheimer's, Parkinsonism) | tau |
| Parkinsonism | tyrosine hydroxylase |
| amyotrophic lateral sclerosis | excitory amino acid transporter 2 |
| schizophrenia | Neuronal NOS |
| schizophrenia | GABAA gamma2 receptor |
| schizophrenia | neural cell adhesion molecule |
| immunodeficiency (SCID) | adenosine deaminase |
| Wagner's disease (vitreoretinal degeneration) | collagen |
| osteoarthritis | collagen |
| osteoporosis | collagen |
| aortic aneurysms | collagen |
| types IV and VII Ehlers-Danlos syndrome | collagen |
| familial arrhythmogenic right ventricular dysplasia | NAPOR (CUGBP2) |
| Pelizaeus-Merzbacher disease | proteolipid M6B |

In some cases the RNA or mRNA will exist in fragmented form when it is collected. In other cases it is preferred desireable to fragment the RNA before contacting it to the array for carrying out the primer extension reaction. Such fragmentation may be carried out by any suitable technique which provides fragments suitable for binding to the primers in the array, e.g., up to 40 or 50 nucleotides in length. RNA can be fragmented by any suitable technique, including chemical and enzymatic techniques, such as by contacting the RNA to ammonia.

Primers used to carry out the present invention are, as noted above, generally oligonucleotide primers, which may be natural or synthetic and may include modified backbones, such as hybrids of DNA and RNA. In general such primers may be 6, 7, 8 or 10 nucleotides in length up to 25, 30, 40 or 50 nucleotides in length, or more. The primers are designed to selectively hybridize to a target or template nucleic acid under a given set of hybridization conditions. The primers may be targeted to a particular exon (that is, hybridize by Watson-Crick pairing to a particular corresponding segment in an exon), or targeted to an exon-exon junction as described further below. The primers are immobilized on a solid support to form an array as discussed further below.

The primer extension reactions used to carry out the present invention may be carried out in accordance with known techniques or modifications thereof which will be apparent to those skilled in the art based upon the disclosure herein. In general, such reactions extend the primer with a labeled or detectable base as described above. Such reactions are known and are, in general, modifications of polymerase chain reaction technology. The reactions typically involve contacting the target or potential template mRNA, pre-processed as may be necessary, to the array. The contacting step is typically carried out by contacting an aqueous solution comprising or containing the sample or template mRNA, along with any necessary reagents for the primer extension reaction (e.g., labeled bases, an RNA-dependent DNA polymerase or reverse transcriptase) to the solid support. The reaction may be carried out with a reverse transcriptase, particularly a reverse transcriptase having a deleted or inactivated RNase H segment or otherwise lacking RNase H activity (e.g., by virtue of introducing a point mutation or deletion into the RNase H domain of a cDNA encoding the protein). Such reverse transcriptases are known and available as, for example, SUPERSCRIPT™ and SUPERSCRIPT™ II reverse transcriptase, or M-MLV Reverse transcriptase RNase H minus (Cat. # M5301), deletion mutant, and M-MLV reverse transcriptase, RNase H minus, point mutant (Cat. # M3682) from Promega Corp. The particular extension reaction format is not critical and the labeled bases may be added singly, added as groups of two or more, added in cyclically repeated extension reactions, etc. Detection of primers extended with the labeled bases is discussed further below. Those skilled in the art will appreciate many variations for the primer extension reaction based upon the teaching herein and established techniques, including but not limited to those described in U.S. Pat. No. 6,280,954 to Ulfendahl, U.S. Pat. No. 6,153,379 to Caskey et al., U.S. Pat. No. 6,004,744 to Goelet et al., U.S. Pat. No. 5,888,819 to Goelet et et al., and U.S. Pat. No. 6,294,336 to Boyce-Jacino et al.

D. Primer Arrays and Apparatus.

The choice of solid support for carrying out the present invention is not critical. Any suitable material can be used, including but not limited to glass, silicon, silicon dioxide, etc., depending upon factors such as the density of primers desired on the array and the procedure employed to immobilize the primers on the array.

Primer arrays or oligonucleotide arrays useful for carrying out the present invention can be prepared by any suitable technique, numerous varieties of which are known to those skilled in the art. One specific example of a suitable technique for preparing such arrays, in which the oligonucleotide primers are attached or bound to the solid support by the 5′ end thereof, is given in U.S. Pat. No. 5,908,926 to Pirrung et al. The primers have a free terminal —OH group for subsequent elongation in the primer extension reaction. In general, the array has, at separate or distinct locations thereon, (a) a plurality (at least 2, 3, 4 or 5 or more) of different primers targeted to particular exons that are potentially in the mRNA, (b) one primer or (preferably) a plurality (e.g., at least 2, 3, 4 or 5 or more) of different primers targeted to different exon-exon junctions that may potentially be in the mRNA, or (c) both (a) and (b) above.

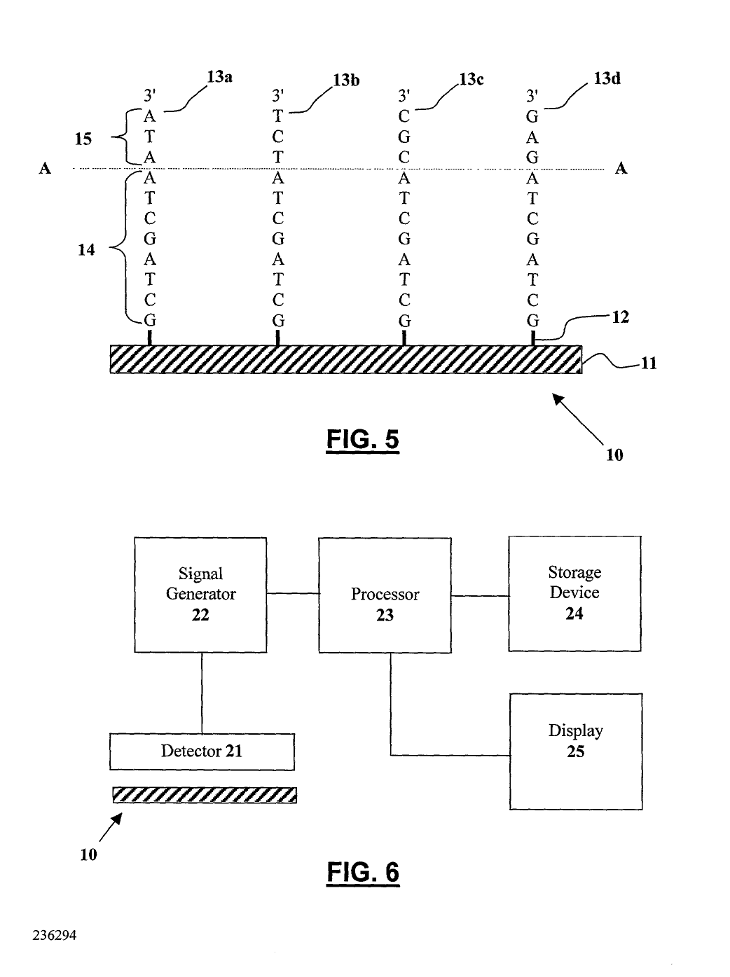

The primers may be of any suitable length, as discussed above. Criteria for designing primers targeted to particular exons are known in the art. When, however, the primers are targeted to exon-exon junctions, particular design criteria may be used. As illustrated in FIG. 5, an array 10 comprises a substrate or solid support 11 having a plurality (in this case four) of different primers 13a, 13b, 13c, 13d immobilized thereto by any suitable technique, such as by covalent linkage 12 (In other embodiments, the primers could be releasably affinity bound to the solid support, etc.). In the embodiment of FIG. 5, all primers are targeted to four different exon-exon junctions, where one of the exons is common to all, and the other exon varies among all. Hence, the portion of all primers below line A-A is a common primer segment in all four primers, and is targeted to the common exon segment. This common primer segment is represented as segment 14 in primer 13a. In contrast, the portion of all primers above line A-A is a variable primer segment, and in all cases is targeted to the variable exon segment. This variable primer segment is represented as segment 15 in primer 13a. Note that the common primer segments are preferably longer than the variable primer segments, and the common primer segments are preferably coupled to the solid support, preferably by the 5′ end thereof. In general, the variable primer segment is preferably from 2, 3 or 4 nucleotides in length to 5, 7 or 10 nucleotides in length (or optionally, though less preferably, more), and the common primer segment is preferably from 4, 5 7 or 8 nucleotides in length up to 20, 30, 40 or 50 nucleotides in length (or optionally, though less preferably, more). The common primer segments thus contribute to the overall affinity of the primers, while the variable primer segments contribute to the selectivity of the primers, particularly at the portion most sensitive to the primer extension reaction (the portion free for elongation).

Apparatus for carrying out the present invention can be implemented in any of a variety of ways, as generally illustrated in FIG. 6. An apparatus for reading an array 10 generally comprises a signal detector 21 positioned for reading or detecting the detectable groups bound to the primers, coupled to signal generator 22 such as an analog to digital converter or a suitable input-output board for a general computer. A processor 23, is operatively associated with the signal generator which processor may be implemented as hardware, software, or combinations of hardware and software. In one embodiment the processor may be a general purposes computer. The “signal generator” may be implemented in part within the hardware and/or software, depending on the particular system employed. A storage device 24 such as a hard drive is typically connected to or associated with the processor for storing collected data, and a display 25 such as a cathode ray tube, liquid crystal display or plasma monitor may be connected to the processor for reading or interpreting data. In use, the signal generator generates the values (preferably digital values and in a particularly preferred embodiment a binary value such as “0” or “1”), which values are then manipulated by the processor to produce an overall code representing the exons present in the mRNA, or an overall code representing the exon-exon junctions present in the sample (from which latter information the particular splice variants within a sample can be determined). The code or codes can then be manipulated in any suitable manner, such as by simply displaying the codes, storing the codes for subsequent data analysis, utilizing the code to indicate the presence of a particular splice variant for diagnostic purposes, etc. It will be appreciated that the code need not be displayed or even stored as the code per se, but can be translated into a symbol or other suitable indicia or representation of the desired information. Those skilled in the art will appreciate many particular ways to implement systems of the present invention in light of the teaching set forth herein, utilizing, for example, the teachings set forth in U.S. Pat. Nos. 6,245,511; 6,215,894; 6,017,496; 5,925,562; 5,751,629; 5,741,462; etc.

The examples which follow are set forth to illustrate the present invention, and are not to be construed as limiting thereof.

### EXAMPLE 1

Materials and Methods

Primers. Initial experiments to analyze splice variants of CD44 RNA by APEX used primers addressing unique sites within each exon v2 through v10. Oligonucleotides were synthesized with 5′-T₁₀ linker sequences, 5′-phosphitylated, and sulfurized with Beaucage reagent. The primers used in APEX experiments are in Table 1.

TABLE 1

| | | |
|---|---|---|
| EXON | PRIMER SEQUENCE | SEQ ID NO |

Exon-speciflc Primers

| | | |
|---|---|---|
| V2 | CTTGCCTCTTGG | SEQ ID NO:1 |
| V3 | CATTTGGCTCCCAGCC | SEQ ID NO:2 |
| V4 | GGTTGTCTGAAGTAGCAC | SEQ ID NO:3 |
| V5 | GTGGGGTCTCTTCTTCC | SEQ ID NO:4 |
| V6 | CCTCATGCCATC | SEQ ID NO:5 |
| V7 | GTTGGTGTTGTCCTTCC | SEQ ID NO:6 |
| V8 | TTGCAGTAGGCTGAAGCG | SEQ ID NO:7 |
| V9 | TATCTTCTTCCAAGCC | SEQ ID NO:8 |
| V10 | CTGGGATGAAGGTCCTGC | SEQ ID NO:9 |

Exon 5 Junction Primers

| | | |
|---|---|---|
| 5-V2 | ACTAGTGCTCATCAAAGTGG | SEQ ID NO:1O |
| 5-V3 | GGTATTTGAAGACGTACTGG | SEQ ID NO:11 |
| 5-V4 | CCGTGGTGTGGTTGAAATGG | SEQ ID NO:12 |
| 5-V5 | GCCATTTCTGTCTACATTGG | SEQ ID NO:13 |
| 5-V6 | ACTAGGAGTTGCCTGGATGG | SEQ ID NO:14 |
| 5-V7 | GGTATGAGCTGAGGCTGTGG | SEQ ID NO:15 |
| 5-V8 | ATGACTGGAGTCCATATTGG | SEQ ID NO:16 |
| 5-V9 | CTGAGAATTACTCTGCTTGG | SEQ ID NO:17 |
| 5-V10 | TGTGACATCATTCCTATTGG | SEQ ID NO:18 |
| 5-16 | GAATGTGTCTTGGTCTCTGG | SEQ ID NO:19 |

Exon 16 Junction Primers

| | | |
|---|---|---|
| V2-16 | TGTGTCTTGGTCTCCAGCCA | SEQ ID NO:20 |
| V3-16 | TGTGTCAAGGTCTCTGGTGC | SEQ ID NO:21 |
| V4-16 | GAATGTGTCTTGGTCTCCAG | SEQ ID NO:22 |
| V5-16 | GAATGTGTCTTGGTCTCTTG | SEQ ID NO:23 |
| V6-16 | AATGTGTCTTGGTCTCCAGC | SEQ ID NO:24 |
| V7-16 | GAATGTGTCTTGGTCTCCCA | SEQ ID NO:25 |
| V8-16 | AATGTGTCTTGGTCTCGCGT | SEQ ID NO:26 |
| V9-16 | GAATGTGTCTTGGTCTCTGC | SEQ ID NO:27 |
| V10-16 | AATGTGTCTTGGTCTCCTGA | SEQ ID NO:28 |

Neighboring Exon Primers

| | | |
|---|---|---|
| V2-V3 | ATTTGAAGACGTACCAGCCA | SEQ ID NO:29 |
| V3-V4 | TGGTGTGGTTGAAATGGTGC | SEQ ID NO:30 |
| V4-V5 | GCCATTTCTGTCTACATCAG | SEQ ID NO:31 |
| V5-V6 | ACTAGGAGTTGCCTGGATTG | SEQ ID NO:32 |
| V6-V7 | GTATGAGCTGAGGCTGCAGC | SEQ ID NO:33 |
| V7-V8 | ATGACTGGAGTCCATATCCA | SEQ ID NO:34 |
| V8-V9 | CTGAGAATTACTCTGCTGCG | SEQ ID NO:35 |
| V9-V10 | TGTGACATCATTCCTATTGC | SEQ ID NO:36 |

Microarray Preparation. In preparation for spotting of microarrays, slides were first cleaned and then silanized. Slides were cleaned with 1M KOH and 1% Decon, for 30 min at 60° C., rinsed with distilled water five times, cleaned with 1 M HCl in ethanol for 30 min at room temperature, and rinsed again with distilled water five times. The slides were then allowed to dry for 2-3 hours at 100° C. The slides were subsequently silanized with 1% N-(3-diethylmethylsilylpropyl) bromoacetamide [DiOEt] in ethanol for 5 min and then allowed to dry for 1 hour at 100° C. The slides were next washed with acetone and again allowed to dry for 1-2 hours at 100° C.

Microarrays were prepared by spotting the resulting 5′-phosphorothioate primers (40-100 μM in 0.5 M carbonate buffer, pH 9) onto bromoacetamide glass using our previously described method (Pirrung, et al. (2000) Langmuir 16:2185). Briefly, 30 nL of each oligonucleotide was spotted onto the silanized glass using a micropipette and allowed to completely dry for approximately 10 min. Spotted slides were stored in a humid chamber for 20-30 min and then washed with distilled water.

RNA Preparation. The CD44 cDNA used to prepare the RNA templates contains all variable exons v2-v10 (Grünthert (1993) Curr. Top. Microbiol. Immunol 184:47-63). PCR was used to make the cDNA template. Complementary reverse primers (in exon 16 or v6) and forward primers (in exon 5 or v6) bearing T7 RNA polymerase promoters at their 5′-ends were used for the amplification (see Table 2).

TABLE 2

| | | |
|---|---|---|
| PRIMER | SEQUENCE | SEQ ID NO |

Forward Primers

| | | |
|---|---|---|
| T7-ES | TTGTAATACGACTCACTATAGGGACAGTCCC | SEQ ID NO:37 |
| | TGGATCACC | |
| T7-EV6 | TTGTAATACGACTCACTATAGGGCAACTCCT | SEQ ID NO:38 |
| | AGTAGTACAACGG | |

Reverse Primers

| | | |
|---|---|---|
| R-E16 | GTTTGCTCCACCTTCTTGACTCCC | SEQ ID NO:39 |
| R-V6 | GTACTACTAGGAGTTGCCT | SEQ ID NO:40 |

T7 RNA polymerase promoters are underlined.

PCR amplification conditions were 94° C. for 30 sec, 55° C. for 60 sec, and 72° C. for 30 sec for 25 cycles. PCR products were purified using Wizard® PCR Preps DNA Purification System (Promega).

In vitro transcription was conducted using the T7 Transcription Kit (Epicentre). RNA was subsequently purified by adding 1/10 volume 3 M sodium acetate, pH 5, and an equal volume of phenol/chloroform solution. RNA sample was mixed well and centrifuged for 3 min. To the supernatant was then added 2.5 volumes of ethanol and again the sample was centrifuged for 3 min. The supernatant was discarded. The pellet was washed with absolute ethanol and then dissolved in distilled water.

Partial hydrolysis of the RNA was conducted by adding an equal volume of conc. ammonium hydroxide and then placing the RNA in a heating block for 3 min at 70° C. The sample was then evaporated completely in a Speed Vac and redissolved in distilled water. This resulted in an average template length of 40 nt based on agarose gel electrophoresis.

Hybridization. The solution used for hybridization was approximately 1 μg of partially hydrolyzed RNA template dissolved to 4× (final) SSC buffer. The RNA was allowed to anneal for 2 min at 90° C. and then cooled to 30° C. over 5 min on the thermal cycler. The hybridization mix was then discarded.

Single Nucleotide Extension. The APEX reaction mix includes 1×Reverse Transcription buffer, 10 mM DTT, 18 μM ddNTP (minus T), 20 pM fluorescein-ddUTP, 0.4 M trehalose, 10 U RNAse inhibitor, and 400 Units RNAseH(−) MMLV reverse transcriptase in a total volume of 50 μL. Reaction mix was exposed to the array under a cover well and held at 37° C. for 10-20 min. The use of the RNAse H(−) RT and the fragmentation step were crucial to the success of RNA APEX. Similar observations at the fragmentation step have been made in microarray hybridization of cRNA (Chee et al. (1996) Science 274:610-4).

Detection. The array was washed with water and then a drop of 25 mM Na₂CO₃ buffer, pH 10 was placed on the slide. The array was covered with a coverslip and scanned at 488 nm by confocal fluorescence microscopy (8-bit dynamic range; BioRad MRC-1000).

### EXAMPLE 2

Validation of RNA APEX Analysis of CD44 mRNA

With an RNA template, including each of the nine variable exons, APEX signals were seen for all primers (FIG. 2, Panel A). A template bearing only exons v6-v10 gave an average APEX S/B of 12-20 at primers addressing exons included in the template, and of <1 at primers addressing exons absent from the template (FIG. 2, Panel B). Likewise, the template bearing only exons v2-v5 gave an average APEX S/B of 12-18 at primers addressing included exons, and of <1 at primers addressing absent exons (FIG. 2, Panel C). These data established the validity of RNA APEX for exon-specific analysis of CD44 mRNA. In Boolean codes for the variable region v2-v10, the first template was 111 111 111, the second was 000 011 111, and the third was 111 100 000. These are “splicotypes” for this locus.

### EXAMPLE 3

RNA APEX Microarray Assay for Variant Splice Forms of CD44 in Breast Cancer

Many human diseases are related to RNA splicing (Philips and Cooper (2000) Cell. Mol. Life Sci. 57:235-49; Cooper and Mattox (1997) Am. J. Hum. Genet. 61:259-66). Variant splicing and up-regulation of the expression of CD44, which generally relate to a poor prognosis, can occur in neoplastic disease (Sneath and Mangham (1998) Mol. Pathol. 51b:191-200; Zoller (1995) J. Mol. Med. 73:425-38; Gunthert, et al. (1995) Cancer Surv. 24:19-42). The functional reasons for this observation seem to relate to enhancement of metastatic motility (Ponta, et al. (1994-95) Invasion Metastasis 14:82-6) and adhesion power of transformed cells. The translation products of the variant exons may affect the activity of the cell adhesion molecule. One particular CD44 antigen, v6, is found in many breast (Herrera-Gayol and Jothy (1999) Exp. Mol. Pathol. 66:149-56), bladder (Cooper (1995) J. Pathol. 177:1-3), and colon (Herrlich, et al. (1995) Eur. J. Cancer 31A:1110-2) cancers. Alternative splicing of the CD44 mRNA is due not to genomic changes but to transacting splicing factors (SR proteins (Smith and Valcárcel (2000) Trends Biochem. Sci. 25:381-8; Graveley (2000) RNA 6:1197-211). CD44 has been examined as a molecular diagnostic in cancer. Protein levels (Woodman, et al. (2000) Clin. Cancer Res. 6:2381-92) or RNA expression levels (Goodison, et al. (1997) Cancer Res. 57:3140-4) have been studied, but the absence of variant-specific reagents has limited the data that can be obtained and the conclusions that can be drawn. Therefore, an examination of the utility of the RNA APEX microarray assay for variant splice forms of CD44 in breast cancer was conducted.

Total cDNA was obtained (Biochain, Inc.) from the primary breast tumor (including connective and normal breast tissue) of a 60-year old patient. The variable CD44 locus was amplified by two rounds of PCR. The first round was conducted using Pfu polymerase (10× buffer from Stratagene) under the following hot start amplification conditions: 92° C. for 30 sec, 55° C. for 60 sec, and 72° C. for 60 sec for 25 cycles. Total amplicons were isolated and used as template for the second round PCR. This round was performed using Thermosequenase under the following amplification conditions: 92° C. for 30 sec, 55° C. for 60 sec, and 72° C. for 60 sec for 25 cycles. Amplifications were conducted with exon 5 (T7-E5) and reverse exon 16 (R-E16) primers (Table 2).

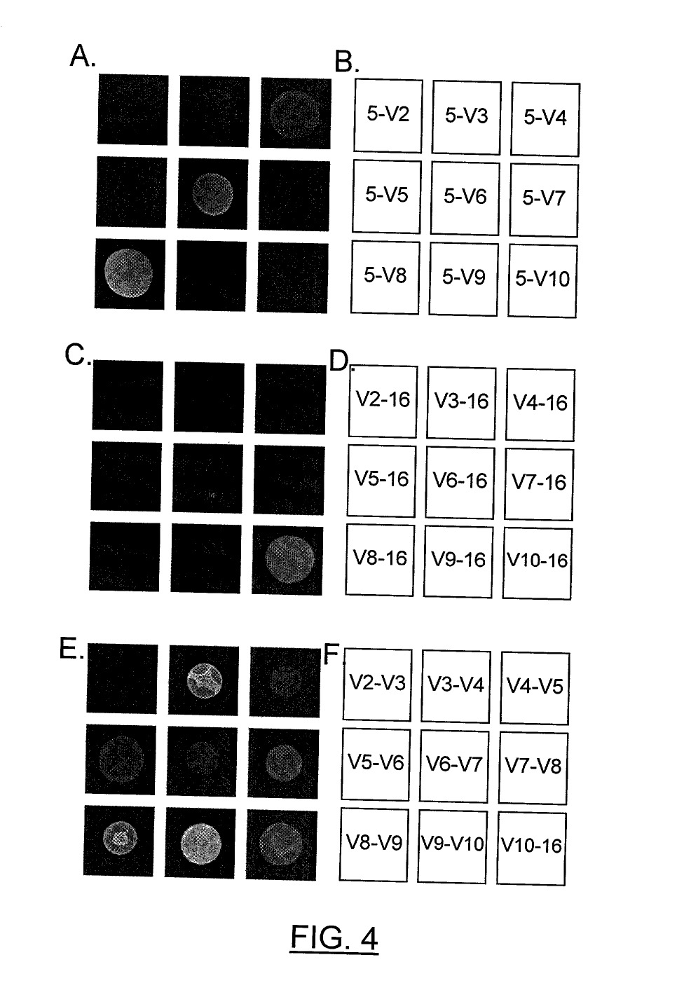

The amplification products were separated on a 1% TAE agarose gel and showed at least 5 bands (FIG. 3). PCR products using the human breast tumor cDNA template were at approximately 1300 bp (8-9 variant exons), 1200 bp (6-7 variant exons), 800 bp (5 variant exons), 550 bp (3 variant exons), and 160 bp (0 variant exons). The 160 bp amplicon is the standard CD44 (without variant exons); the other amplicons represent variant splice forms (van Weering, et al. (1993) PCR Methods Appl. 3:100-6). Analysis of this mixed RNA population (resulting from multiple cell types within the tumor sample) with the exon-specific microarray gave the tentative variant splicotype of 0 - - - 1 (the “-” indicates a mixture of forms), with mixed signals for exons v3-v9. The normally spliced form gave no signal with the variant exon-specific microarray. An additional microarray was prepared with primers specific for the junctions of exon 5 with each variant exon and for the junctions of each variant exon with exon 16. For the latter, only junctions 5-16 (standard) and v10-16 were observed, while the former showed junctions 5-v3, 5-v4, 5-v6, and 5-v8 (FIG. 4). The variant splicotypes in this sample, which were consistent with amplicon sizes, were thereby established as 011 111 111, 001 111 111, 000 011 111, and 000 000 111. Additional, weaker signals were also observed. RNA APEX showed extremely low non-specific signals, suggesting that even these weak signals should be regarded as positives, and that further CD44 splice forms may exist in this tissue (e.g., a v9-16 junction is possible). While in this sample the included exons were contiguous in the genomic sequence, mature mRNAs are known in which non-contiguous exons are joined.

To date, there have been no previous reports of the presence of more than two CD44 isoforms within the same tissue. Most analyses of CD44 variants have relied on immunofluorescence of antigens derived from the variant exons, so it is unsurprising that detection of more than two isoforms simultaneously has been difficult. Such studies also provide only the percentage expression of each exon and cannot identify the specific sequences of multiple variant forms. This microarray method can detect any type of variant among known exons even when multiple forms exist in the same tissue sample. Ligation reactions on arrays similar to those described here (bearing primers with free 3′-ends) should also make possible the identification of unknown exons (Kim, H., unpublished). Application of these methods to analysis of human tumors, tissues, and cell lines should provide a much better picture of the diversity in CD44 splicing.

This RNA APEX analysis method provides a high fidelity, digital interpretation of the presence of variantly spliced exons within an RNA. The APEX signal for individual primers shows no direct relationship to primer concentration in the spotting solution, sequence, Tₘ, or Pur/Pyr ratio, nor to template concentration. As currently constituted, the method does not address the relative amounts of the variant splice forms. With RNA samples containing only one spliced form, splicotypes can be determined using microarrays bearing only exon-specific primers. With heterogeneous RNAs, multiple splicotypes can be assigned using additional splice-specific primers. Applications of this technique should be broad given an extensive database (Stamm, et al. (2000) DNA Cell Biol. 19:739-56) of alternatively spliced exons.

The foregoing examples are illustrative of the present invention, and are not to be construed as limiting thereof. The invention is described by the following claims, with equivalents of the claims to be included therein.

What is claimed is:

1. A method for determining the exons present in a potentially variantly spliced mRNA, said method comprising the steps of:
(a) providing a potentially variantly spliced mRNA, said mRNA encoded by a DNA, said DNA comprising a plurality of exons, each of which plurality of exons may or may not be included in said mRNA;
(b) providing an array, said array comprising a plurality of different primers immobilized on a solid support at distinct locations thereon, with each of said plurality of different primers selectively hybridizing to a corresponding one of said plurality of exons to form a duplex therebetween;
(c) contacting said mRNA to said array so that a duplex is formed between each different primer and each corresponding exon if said corresponding exon is included in said mRNA;
(d) subjecting said duplexes to a primer extension reaction so that the primers in said duplexes are extended with at least one labeled base; and then
(e) detecting the presence or absence of said labeled base in each of said plurality of primers, the presence of said at least one labeled base indicating the presence of the exon to which said primer selectively binds in said potentially variantly spliced mRNA.

2. The method according to claim 1, wherein said mRNA comprises mRNA fragments.

3. The method according to claim 1, further comprising the step of fragmenting said mRNA prior to said contacting step.

4. The method according to claim 1, wherein said primer extension reaction is carried out with a reverse transcriptase having a deleted RNase H segment.

5. The method according to claim 1, wherein said primers are immobilized to said solid support by the 5′ end thereof so that the 3′ ends of said primers are available to be extended in said primer extension reaction.

6. The method of claim 1, wherein said mRNA is provided from a biological sample.

7. The method according to claim 1, wherein said mRNA is produced by polymerization from a corresponding cDNA.

8. The method according to claim 1, wherein said mRNA is CD44 mRNA.

9. A method according to claim 1, wherein said detecting the step is followed by the steps of:
(f) generating a plurality of values, each of said values indicating the presence or absence of each of said exons in said mRNA; and then
(g) generating a code representing the exons present in said mRNA from said plurality of generated values.

10. The method of claim 9, wherein each of said values is a digital value, and said code is a digital code.

11. The method of claim 9, wherein each of said values is a binary value, and said code is a binary code.

12. A method for distinguishing splice variants in a mixed mRNA sample, said method comprising the steps of:
(a) providing a mixed mRNA sample, said mixed mRNA sample comprising a plurality of splice variants, each one of said plurality of splice variants containing a distinct exon-exon junction not found in each other of said plurality of splice variants;
(b) providing an array, said array comprising a plurality of different primers immobilized on a solid support at distinct locations thereon, with each of said plurality of different primers selectively hybridizing to a corresponding one of said distinct exon-exon junctions to form a duplex therebetween;
(c) contacting said mixed mRNA sample to said array so that a duplex is formed between each different primer and each corresponding splice variant if said corresponding splice variant is included in said mixed mRNA sample;
(d) subjecting said duplexes to a primer extension reaction so that the primers in said duplexes are extended with at least one labeled base; and then
(e) detecting the presence or absence of said labeled base in each of said plurality of primers, the presence of said at least one labeled base indicating the presence of the splice variant to which said primer selectively binds in said mixed mRNA sample.

13. The method according to claim 12, wherein said plurality of splice variants comprises at least three splice variants.

14. The method of claim 11, wherein said detecting step (e) is followed by the step of:
(f) determining the presence of at least three distinct splice variants in said sample from said detected presence or absence of said labeled base in each of said plurality of primers.

15. The method according to claim 12, wherein:
a plurality of said splice variants contains an exon-exon junction comprising a common exon segment coupled to a variable exon segment, with said common exon segment being the same among said plurality of splice variants and said variable exon segments being different among said plurality of splice variants;
a plurality of said primers contains a common primer segment coupled to a variable primer segment, with the common primer segment corresponding to said common exon segment and being the same among said plurality of primers, and with said variable primer segment corresponding to said variable exon segment and being different among said plurality of primers; and
said common primer segments are from 8 to 50 nucleotides in length and are positioned at the 5′ end of said primers, and said variable primer segments are from 2 to 7 nucleotides in length and are positioned at the 3′ end of said primers.

16. The method according to claim 12, wherein said mRNA comprises mRNA fragments.

17. The method according to claim 12, further comprising the step of fragmenting said mRNA prior to said contacting step.

18. The method according to claim 12, wherein said primer extension reaction is carried out with a reverse transcriptase having a deleted RNase H segment.

19. The method according to claim 12, wherein said primers are immobilized to said solid support by the 5′ end thereof so that the 3′ ends of said primers are available to be extended in said primer extension reaction.

20. The method of claim 12, wherein said mRNA is provided from a biological sample.

21. The method according to claim 12, wherein said mRNA is produced by polymerization from a corresponding cDNA.

22. The method according to claim 12, wherein said mRNA is CD44 mRNA.

23. A method according to claim 12, wherein said detecting the step is followed by the steps of:
(f) generating a plurality of values, each of said values indicating the presence or absence of each of said exons in said mRNA; and then
(g) generating a code representing the exons present in said mRNA from said plurality of generated values.

24. The method of claim 23, wherein each of said values is a digital value, and said code is a digital code.

25. The method of claim 23, wherein each of said values is a binary value, and said code is a binary code.

26. An array useful for determining the exons present in a potentially variantly spliced mRNA, said mRNA encoded by a DNA, said DNA comprising a plurality of exons, each of which plurality of exons may or may not be included in said mRNA, said array comprising:
a solid support; and
a plurality of different primers immobilized on said solid support at distinct locations thereon, with each of said plurality of different primers selectively hybridizing to a corresponding one of said plurality of exons under predetermined hybridization conditions;
wherein said primers are immobilized to said solid support by the 5′ end thereof;
and wherein said mRNA is CD44 mRNA.

27. An array according to claim 26, wherein said plurality of primers comprise at least three primers.

28. An array according to claim 26, wherein each of said plurality of primers is from 10 to 50 nucleotides in length.

29. An array useful for distinguishing splice variants in a mixed mRNA sample, said mixed mRNA sample comprising a plurality of splice variants, each one of said plurality of splice variants containing a distinct exon-exon junction, each of said distinct exon-exon junctions comprising a common exon segment coupled to a variable exon segment, with said common exon segment being the same among said plurality of splice variants and said variable exon segments being different among said plurality of splice variants, said array comprising:
a solid support; and
a plurality of different primers immobilized on said solid support at distinct locations thereon, with each of said plurality of different primers selectively hybridizing to a corresponding one of said distinct exon-exon junctions under predetermined hybridization conditions to form a duplex therebetween;
wherein each of said plurality of primers contains a common primer segment coupled to a variable primer segment, with said common primer segment corresponding to said common exon segment and being the same among said plurality of primers, and with said variable primer segment corresponding to said variable exon segment and being different among said plurality of primers;
and wherein said common primer segments are from 8 to 50 nucleotides in length and are positioned at the 5′ end of said primers, and said variable primer segments are from 2 to 7 nucleotides in length and are positioned at the 3′ end of said primers.

30. An array according to claim 29, wherein said plurality of primers comprise at least three primers.

31. A system for determining the exons present in a potentially variantly spliced mRNA with a plurality of different primers, each of said plurality of primers selectively hybridizing to a corresponding one of said plurality of exons, and each of said plurality of primers being extended with at least one labeled base when said corresponding one of said plurality of exons is present, said system comprising:
(a) a detector for detecting the presence or absence of said labeled base from each of said plurality of primers;
(b) a signal generator operatively associated with said detector for generating a plurality of values, each of said values indicating the presence or absence of each of said exons in said mRNA from said detected presence or absence of said labeled base from each of said plurality of primers; and
(c) a processor operatively associated with said signal generator for generating a code representing the exons present in said mRNA from said plurality of generated values.

32. A system according to claim 32, further comprising a storage device operatively associated with said processor for storing said code.

33. A system according to claim 32, further comprising a display operatively associated with said processor for displaying said code.

34. A system according to claim 32, wherein each of said values is a digital value, and said code is a digital code.

35. A system according to claim 32, wherein each of said values is a binary value, and said code is a binary code.

36. A system for distinguishing splice variants in a mixed mRNA sample with a plurality of different primers, each of said plurality of primers selectively hybridizing to a distinct exon-exon junction in each of said plurality of splice variants, and each of said plurality of primers being extended with at least one labeled base when said corresponding one of said plurality of exons is present, said system comprising:
(a) a detector for detecting the presence or absence of said labeled base from each of said plurality of primers;
(b) a signal generator operatively associated with said detector for generating a plurality of values, each of said values indicating the presence or absence of each of said splice variants in said mixed mRNA sample from said detected presence or absence of said labeled base from each of said plurality of primers; and
(c) a processor operatively associated with said signal generator for generating a determination of the splice variants present in said mixed mRNA sample from said plurality of generated values.

37. A system according to claim 36, further comprising a storage device operatively associated with said processor for storing said determination of the splice variants present in said mixed mRNA sample.

38. A system according to claim 36, further comprising a display operatively associated with said processor for displaying said determination of the splice variants present in said mixed mRNA sample.

39. A system according to claim 36, wherein each of said values is a digital value.

40. A system according to claim 36, wherein each of said values is a binary value.
